(19) United States
(12) Patent Application Publication
(10) Pub. No.: US 2020/0129123 A1
(43) Pub. Date: Apr. 30, 2020

(54) GARMENT, BIOLOGICAL INFORMATION MEASUREMENT METHOD AND BIOLOGICAL INFORMATION MEASUREMENT SYSTEM

(71) Applicant: KYOCERA Corporation, Kyoto (JP)

(72) Inventor: Hiromi AJIMA, Kawasaki-shi, Kanagawa (JP)

(73) Assignee: KYOCERA Corporation, Kyoto (JP)

(21) Appl. No.: 16/478,288

(22) PCT Filed: Jan. 12, 2018
(86) PCT No.: PCT/JP2018/000700

§ 371 Date: Jul. 16, 2019

(30) Foreign Application Priority Data
Feb. 1, 2017 (JP) 2017-016955

(51) Int. Cl. A61B 5/00; A61B 5/16; A61B 5/0205

(52) U.S. Cl. CPC A61B 5/6804; A61B 2562/0219; A61B 5/0205; A61B 5/165

(57) ABSTRACT

Provided is a garment including a gyro sensor configured to detect a change in a wearer's abdomen and a controller configured to measure biological information of the wearer based on the change detected.

### CROSS REFERENCE TO RELATED APPLICATION

The present application claims priority to and the benefit of Japanese Patent Application No. 2017-016955 filed on Feb. 1, 2017, the entire contents of which are incorporated herein by reference.

### TECHNICAL FIELD

The present disclosure relates to a garment, a biological information measurement method and a biological information measurement system.

### BACKGROUND

Electronic devices configured to measure the biological information from a measured part such as a wrist or the like of a subject are known. For example, electronic devices that are mounted on a wrist of a subject and measure a pulse of the subject are known.

### SUMMARY

### Solution to Problem

A garment according to an embodiment includes a gyro sensor configured to detect a change in a wearer's abdomen and a controller configured to measure biological information of the wearer based on the change detected.

In a biological information measurement method according to an embodiment, a change in abdomen of a wearer of a garment is detected by a gyro sensor provided to the garment, and biological information of the wearer is measured based on the change detected.

A biological information measurement system according to an embodiment includes a garment including a gyro sensor configured to detect a change in a wearer's abdomen and an external apparatus including a controller configured to measure biological information of the wearer based on the change detected.

### BRIEF DESCRIPTION OF THE DRAWINGS

In the accompanying drawings:

FIG. 1 is a functional block diagram illustrating a schematic configuration of a biological information measurement apparatus provided to a garment according to an embodiment;

FIG. 2 is a diagram schematically illustrating an aorta in the human body;

FIG. 3A is a diagram illustrating an example of a state in which a measured part abuts an abutment;

FIG. 3B is a diagram illustrating an example of a state in which a measured part abuts an abutment;

FIG. 4 is a schematic diagram for illustrating the measurement processing of the pulse wave by the biological information measurement apparatus illustrated in FIG. 1;

FIG. 5 is a flowchart illustrating a procedure of the measurement processing of the pulse wave by the biological information measurement apparatus illustrated in FIG. 1;

FIG. 6 is a diagram illustrating an example of the pulse wave acquired by a sensor;

FIG. 7 is a diagram illustrating a change in the calculated AI over time;

FIG. 8 is a diagram illustrating a measurement result of the calculated AI and a blood glucose level;

FIG. 9 is a diagram illustrating a relationship between the calculated AI and the blood glucose level;

FIG. 10 is a diagram illustrating a measurement result of the calculated AI and neutral fat level;

FIG. 11 is a flowchart illustrating a procedure of estimating the blood fluidity and status of glucose metabolism and lipid metabolism;

FIG. 12 is a diagram illustrating a configuration example of a garment according to an embodiment;

FIG. 13 is a diagram illustrating another configuration example of a garment according to an embodiment;

FIG. 14A is a diagram illustrating still another configuration example of a garment according to an embodiment;

FIG. 14B is a diagram illustrating still another configuration example of a garment according to an embodiment; and

FIG. 15 is a diagram illustrating a schematic configuration of a biological information measurement system according to an embodiment.

### DETAILED DESCRIPTION

It is useful to easily measure the biological information of a person wearing a garment. The present disclosure relates to easy measurement of the biological information of a wearer of a garment. According to a garment, a biological information measurement method and a biological information measurement system of the present disclosure, the biological information of a wearer of a garment can be easily measured. An embodiment will be described in detail below with reference to drawings.

A garment according to an embodiment includes a biological information measurement apparatus configured to measure the biological information of a wearer of the garment. The wearer of the garment can measure one's own biological information by the biological information measurement apparatus with the garment on. The garment according to the present embodiment may have a variety of configurations described below. First, the biological information measurement apparatus provided to the garment according to the present embodiment will be described. Hereinafter, a wearer of the garment according to the present embodiment is appropriately referred to as a “subject” whose biological information is measured by the biological information measurement apparatus provided to the garment.

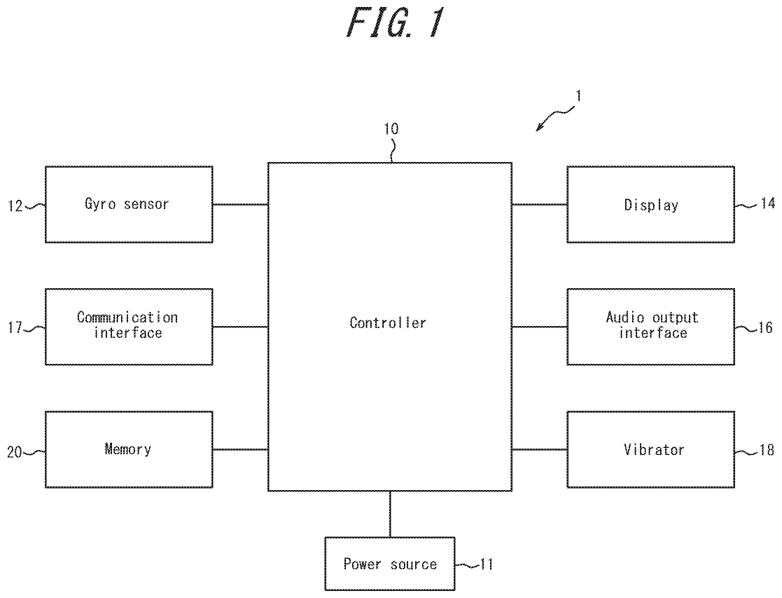

FIG. 1 is a functional block diagram illustrating a schematic configuration of a biological information measurement apparatus provided to the garment according to an embodiment. As illustrated in FIG. 1, a biological information measurement apparatus 1 includes a controller 10, a power source 11, a gyro sensor 12, a display 14, an audio output interface 16, a communication interface 17, a vibrator 18 and a memory 20.

The controller 10 includes a processor configured to control and manage the whole biological information measurement apparatus 1 including each function block thereof. The controller 10 includes a processor such as a central processing unit (CPU) configured to execute a program that stipulates control procedures and a program that measures the biological information of the subject. Such programs are stored in a storage medium such as a memory 20 or the like.

The power source 11 includes a battery, and supplies power to each portion of the biological information measurement apparatus 1. The biological information measurement apparatus 1 receives power supply from the power source 11 or from an external power source during operation. The power source 11 may also receive power supply from outside via a power source line and supply power supplied via the power source line to each portion of the biological information measurement apparatus 1.

The gyro sensor 12 detects the angular velocity of the biological information measurement apparatus 1, and thus detects the displacement of the biological information measurement apparatus 1 as a motion factor. The gyro sensor 12 is a three-axis oscillation gyro sensor that detects the angular velocity on the basis of deformation of a structure by the Coriolis force acting on an oscillating arm, for example. In this context, the structure may be made of materials such as crystal or piezoelectric material such as piezoelectric ceramics and the like. The gyro sensor 12 may also be formed of a material such as silicon by using a micro electro mechanical systems (MEMS) technology. The gyro sensor 12 may also be a gyro sensor of other type such as an optical gyro sensor. The controller 10 can measure the orientation of the biological information measurement apparatus 1 by time-integrating the angular velocity acquired by the gyro sensor 12 once.

The gyro sensor 12 is an angular velocity sensor, for example. However, the gyro sensor 12 is not limited to an angular velocity sensor. The gyro sensor 12 may detect the angular displacement, which is a motion factor, of the biological information measurement apparatus 1. The gyro sensor 12 may detect the motion factor which is processed as a self control factor. The motion factor detected by the gyro sensor 12 is sent to the controller 10.

The controller 10 acquires the motion factor from the gyro sensor 1:2. The motion factor includes an index indicating a displacement of the biological information measurement apparatus 1 based on the pulse at the measured part of the subject. The controller 10 generates a pulsation of the subject on the basis of the motion factor. The controller 10 measures the biological information on the basis of the pulsation of the subject. The measurement processing of the biological information by the controller 10 will be described later.

The display 14 includes a display device such as a liquid crystal display, an organic electro-luminescence panel or an inorganic electro-luminescence panel and the like. The display 14 displays characters, images, symbols, figures and the like. The display 14 may also be a touch screen display that has not only a display function but also a touch screen function. In this case, the touch screen detects a touch by a finger of a wearer and the like or a stylus pen. The touch screen can detect positions thereon touched by fingers or a stylus pen. Detection types of a touch screen include a capacitance type, a resistive film type, a surface elastic wave type (or an ultrasonic wave type), an infrared type, an electromagnetic type, a load detection type and the like. The capacitance type touch screen can detect a touch and/or proximity of a finger, a stylus pen and the like.

The audio output interface 16 notifies a wearer and the like of information through sound output. The audio output interface 16 may be constituted by any speaker and the like. The audio output interface 16 outputs audio signals sent from the controller 10 as sound.

The communication interface 17 sends/receives various kinds of data through wired or wireless communication with an external apparatus. The communication interface 17 can send measurement results of the biological information measured by the biological information measurement apparatus 1 to an external apparatus, for example. The communication interface 17 can also communicate with an external apparatus that stores the biological information of the subject (wearer) to care for the health thereof.

The vibrator 18 notifies the wearer of the information through generation of vibration and the like. The vibrator 18 provides a tactile sensation to the wearer of the biological information measurement apparatus 1 through generation of vibration at any portion of the biological information measurement apparatus 1. As the vibrator 18, any member such as an eccentric motor, a piezoelectric element (a piezo element) or a linear vibrator may be adopted as far as it generates vibration.

The memory 20 stores a variety of programs including application programs and data. The memory 20 may include any non-transitory storage medium such as a semiconductor storage medium, a magnetic storage medium and the like. The memory 20 may include a plurality of types of storage media. The memory 20 may include a combination of a portable storage medium, such as a memory card, an optical disc, or a magneto-optical disc, and an apparatus for reading the storage medium. The memory 20 may include a storage device used as a volatile storage area, such as a random access memory (RAM). The memory 20 stores a variety of information, programs for causing the biological information measurement apparatus 1 to operate, and the like, and also functions as a working memory. The memory 20 may store data detected by the gyro sensor 12 and the measurement result of the biological information, for example.

The biological information measurement apparatus 1 according to an embodiment is not limited to the configuration illustrated in FIG. 1. A main component of the biological information measurement apparatus 1 according to an embodiment is the gyro sensor 12. Thus, in the biological information measurement apparatus 1 according to an embodiment, components except the main component may be omitted or added as necessary. When the biological information measurement apparatus 1 according to an embodiment measures the biological information, a controller may be used, the controller being configured to measure the biological information of the subject on the basis of a change in a measured part of the subject detected by the gyro sensor 12. When the biological information measurement apparatus 1 provided with no controller 10 measures the biological information, the signal detected by the gyro sensor 12 may be sent to an external controller for processing. In the garment provided with the biological information measurement apparatus 1, the controller 10, the display 14, the audio output interface 16, the vibrator 18 and the like may be provided at a position different from a position where the biological information measurement apparatus 1 is disposed,

The biological information measurement apparatus 1 according to the present embodiment is mounted to a variety of types of garments such as, for example, tops such as vests or jackets, bottoms such as pants (trousers) and belts that support bottoms such as pants (trousers). The biological information measurement apparatus 1 is mounted to the above described garment and measures the biological information at a predetermined part (a measured part) of a wearer, that is, a subject. The measured part is a part abutting the wearer when the biological information of the wearer is measured by the garment.

The biological information measured by the biological information measurement apparatus 1 includes at least one of blood component, pulse wave, pulse and pulse wave transmitting velocity, for example. The blood component includes glucose metabolism status and lipid metabolism status, for example. The glucose metabolism status includes a blood glucose level, for example. The lipid metabolism status includes a lipid level, for example. The lipid level includes neutral fat, total cholesterol, high-density lipoprotein (HDL) cholesterol, and low-density lipoprotein (LDL) cholesterol. The biological information measurement apparatus 1 acquires a pulse wave of the subject as the biological information, for example, and measures the biological information such as blood component and the like on the basis of the acquired pulse wave.

Next, the measurement processing of the biological information by the biological information measurement apparatus 1 will be described. The biological information measurement apparatus 1 acquires a motion factor in a state in which an abutment provided on a surface abutting the subject abuts the measured part, and measures the biological information on the basis of the acquired motion factor. The biological information measurement apparatus 1 may acquire a motion factor in a state in which a support provided on the abutting surface abuts the subject at a position different from that of the measured part. In the present embodiment, the measured part may be an abdomen of the wearer (subject), for example. In this case, the gyro sensor 12 of the biological information measurement apparatus 1 detects a change in the abdomen of the wearer.

Upon measurement of the biological information, when the subject wears the garment according to the present embodiment, for example, the biological information measurement apparatus 1 is put into a state in which the measurement processing of the biological information can be performed. The state in which the measurement processing of the biological information can be performed is a state in which an application for measuring the biological information is activated.

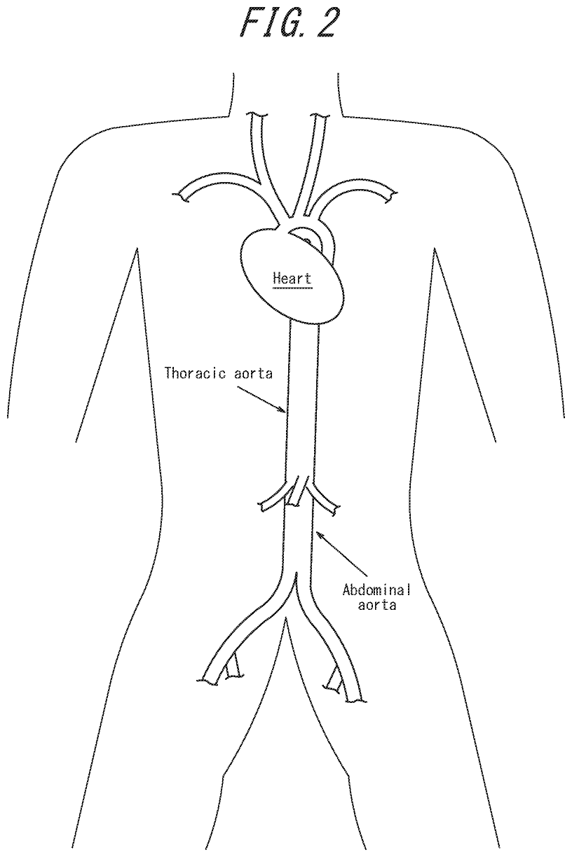

Next, a principle of measurement of the biological information of the subject by the biological information measurement apparatus 1 will be further described below. The biological information measurement apparatus 1 measures the biological information on the basis of a change in the measured part of the subject. FIG. 2 schematically illustrates an internal structure of the human body. FIG. 2 schematically illustrates a part of the internal structure of the human body. In particular, FIG. 2 schematically illustrates a heart and a part of aorta in the human body.

In the human body, the blood pumped out of the heart is carried to each part of the human body through blood vessels. As illustrated in FIG. 2, in the human body, a part of the blood pumped out of the heart passes through the abdomen aorta after passing through the thoracic aorta. Pumping blood out of the heart to the thoracic aorta or the abdomen aorta causes a change such as contraction of the blood vessels. Such change is transmitted throughout the body of the subject and causes a change in predetermined parts, such as a thoracic part, an abdomen part, a femoral part and a wrist, of the subject. Therefore, the gyro sensor 12 can detect a change in a predetermined part of the subject with the biological information measurement apparatus 1 pushed against the predetermined part of the subject. In this manner, the gyro sensor 12 detects a motion factor caused by a change in a predetermined part of the subject.

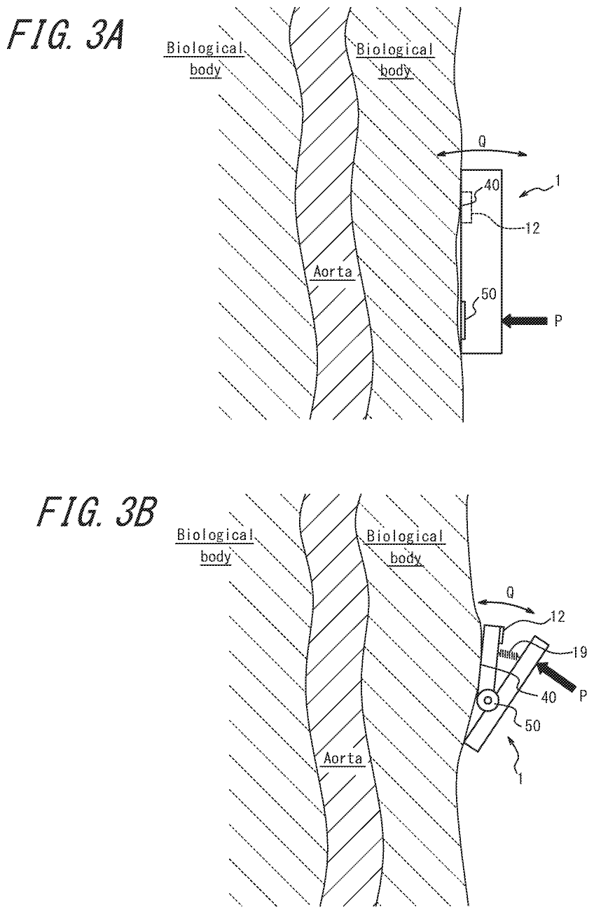

FIG. 3 illustrates an example of an aspect of acquisition of a motion factor by the biological information measurement apparatus 1.

FIGS. 3A and 3B are cross sectional views illustrating a part including an aorta in a biological body such as a human body. FIGS. 3A and 3B illustrate a state in which the abutting surface of the biological information measurement apparatus 1 abuts the measured part of the surface (skin) of the biological body. In this manner, as illustrated in FIGS. 3A and 3B, an abutment 40 and a support 50 provided on the abutting surface of the biological information measurement apparatus 1 abut respectively the measured parts on the surface (skin) of the biological body. In an embodiment, the measured part on the surface of the biological body is defined as a trunk of the subject. The aorta illustrated in FIGS. 3A and 3B may be the thoracic aorta illustrated in FIG. 2 or an abdomen aorta. The aorta illustrated in FIGS. 3A and 3B may also be a femoral artery, a radial artery or an ulnar artery.

As illustrated in FIGS. 3A and 3B, the abutment 40 of the biological information measurement apparatus 1 is pushed against a predetermined part of the subject, On the back side of the abutment 40 is provided with the gyro sensor 12, and by means of the gyro sensor 12, the biological information measurement apparatus 1 acquires a displacement of the biological information measurement apparatus 1 as a motion factor. As illustrated in FIGS. 3A and 3B, the abutment 40 abuts the measured part in a state in which the biological information measurement apparatus 1 abuts a predetermined part of the subject. Further, as illustrated in FIGS. 3A and 3B, in a state in which the biological information measurement apparatus 1 acquires a motion factor, the support 50 abuts the subject at a position different from that of the abutment 40.

As illustrated in FIGS. 3A and 3B, when the biological information measurement apparatus 1 is pushed against the subject so as to abut the subject, the biological information measurement apparatus 1 is displaced in response to vasodilatation/vasoconstriction induced by pulsation of the subject. The biological information measurement apparatus 1 is displaced about the support 50 as a pivot such that the upper end side rotates as illustrated by an arrow Q in FIGS. 3A and 3B. Such displacement is usually a displacement like oscillation in which reciprocating partial rotational movement is repeated. The gyro sensor 12 provided to the biological information measurement apparatus 1 acquires a pulse wave of the subject by detecting a displacement of the biological information measurement apparatus 1. The pulse wave is a change in volume of a blood vessel over time caused by flowing-in of blood, and is taken as a waveform from a surface of the body.

In this manner, in the biological information measurement apparatus 1 according to an embodiment, the gyro sensor 12 detects a motion factor caused by a change in a predetermined part (a measured part) of the subject. The gyro sensor 12 detects a motion factor caused by a change in the predetermined part of the subject in a state in which the biological information measurement apparatus 1 is pushed against the predetermined part of the subject. The controller 10 then performs measurement processing of the biological information of the subject on the basis of the motion factor detected by the gyro sensor 12 as described above.

In this context, examples of the measured parts include a thoracic part, an abdomen part, a femoral part and a wrist. In FIGS. 3A and 3B, as an example of a change in the measured part, a change caused by movement of the blood vessel of the subject is illustrated. However, it is not restrictive, and examples of the change in the measured part of the subject may include not only a change caused by the movement of the blood vessel of the subject, but also at least one of a change caused by breathing of the subject and a change caused by body motion of the subject. Examples of blood vessel of the subject may also include an aorta of the subject. Further, examples of the aorta of the subject may include at least one of an abdomen aorta, a thoracic aorta, a femoral artery, a radial artery and an ulnar artery of the subject. A lot of blood flows continuously in large blood vessels such as aorta. Thus, in the biological information measurement apparatus 1, when an aorta of a subject is determined as an object to be measured, the biological information can be measured stably with high accuracy.

Further, as illustrated in FIG. 3B, when the gyro sensor 12 is pushed against the measured part of the subject via an elastic member 19, it can easily follow a change in the measured part of the subject. As a result, the biological information measurement apparatus 1 can measure the biological information stably with high accuracy. In this context, the elastic member 19 may be any member that generates an elastic force, and may be one that uses spring, rubber, flexible resin, hydraulic pressure, pneumatic pressure, water pressure and the like. The support 50 illustrated in FIG. 3B connects a housing provided with the gyro sensor 12 and a housing provided with no gyro sensor 12. As illustrated in FIG. 3B, the housing provided with the gyro sensor 12 can pivot about the support 50 as an axis with respect to the housing provided with no gyro sensor 12,

The biological information measurement apparatus 1 provided with the gyro sensor 12 enables the subject to measure the biological information from over his/her garment with the garment put on. In other words, the biological information measurement apparatus 1 will make it unnecessary for the subject to take off a garment when the biological information is measured. The biological information measurement apparatus 1 will also make it unnecessary for the subject to bring his/her skin to directly contact with the measurement apparatus, In this manner, when the biological information measurement apparatus 1 is provided to a variety of kinds of garments such as tops (jackets or upper wears), bottoms (lower wears) and belts, the wearer can easily measure the biological information with such garment put on.

Since a conventional acceleration sensor has a large noise, it is hard to say that such acceleration sensor is suitable for the use as a pulse wave sensor. In particular, when a sensor is used for measuring low frequencies of about 1 Hz such as pulse wave and breathing, a compact acceleration sensor installed in a compact measurement apparatus is not commonly used. A relatively large acceleration sensor is usually required for the above purpose.

On the other hand, in the biological information measurement apparatus 1, the gyro sensor 12 is used for measuring the biological information. The gyro sensor generally has a small noise during measurement. The gyro sensor vibrates all the time (in the case of a vibration sensor), thus it can structurally reduce a noise. In the biological information measurement apparatus 1 according to an embodiment, the gyro sensor 12 that can be installed in a compact housing can be adopted.

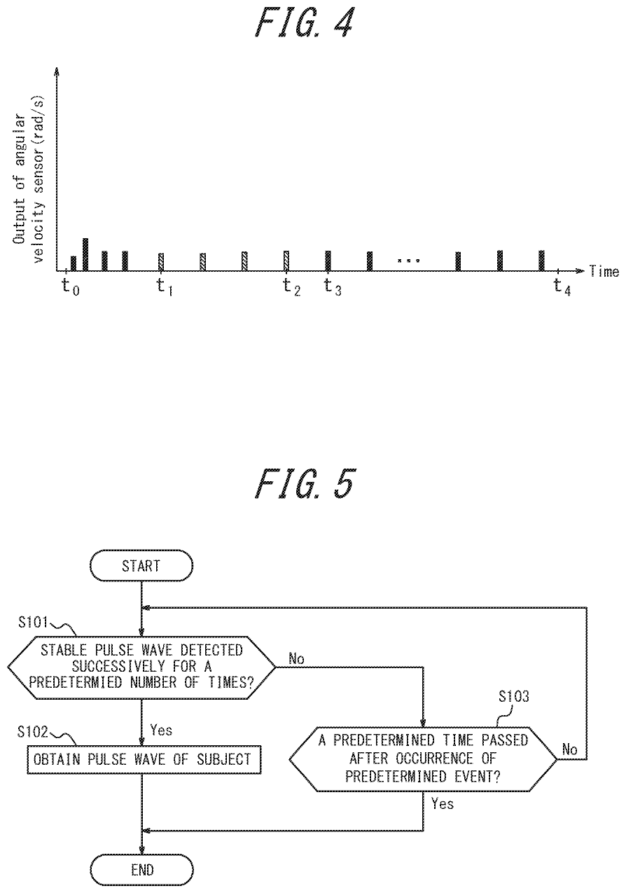

The biological information measurement apparatus 1 performs measurement processing of pulse wave with the abutment 40 abutted a measured part. FIG. 4 is a schematic diagram illustrating the measurement processing of pulse wave by the biological information measurement apparatus 1. FIG. 5 is a flowchart illustrating a procedure of the measurement processing of pulse wave by the biological information measurement apparatus 1. In FIG. 4, the horizontal axis represents the time and the vertical axis schematically represents the output (rad/sec) on the basis of the pulse wave of an angular velocity sensor, which is the gyro sensor 12. In FIG. 4, the output of the angular velocity sensor represents only peaks of respective pulse waves.

Suppose that a predetermined event for the biological information measurement apparatus 1 to start the pulse wave measurement processing occurs at time t₀. Examples of such event include the case in which the wearer of a garment according to the present embodiment wears the garment. Occurrence of such event allows the abutment 40 of the biological information measurement apparatus 1 to abut the measured part of the wearer, which is a subject. Suppose that the biological information measurement apparatus 1 is put into a state in which the measurement processing of the biological information can be performed at time t₀ and starts the measurement processing of pulse wave.

In the biological information measurement apparatus 1, when the pulse wave measurement processing is started, the controller 10 detects output of the gyro sensor 12 in response to the pulsation of the blood vessel of the subject. During a predetermined period of time immediately after the measurement is started (from time t₀ to time t₁ in FIG. 4), output of the gyro sensor 12 is unstable due to adjustment and the like of a position where the abutment 40 abuts the measured part. The pulse wave cannot be acquired correctly in this period. Thus the biological information measurement apparatus 1 may not use the pulse wave measured in this period for measuring the blood component, which is the biological information, for example. The biological information measurement apparatus 1 may not store the pulse wave measured in this period in the memory 20.

The controller 10 determines whether or not it continuously detects stable pulse wave for a predetermined number of times after the pulse wave measurement processing is started (step S101 in FIG. 5). Although the predetermined number of times is four times in the example illustrated in FIG. 4, this is not restrictive. The stable pulse wave means, for example, a variance in peak outputs of each pulse wave and/or a variance in interval between peaks of each pulse waves is/are within a predetermined error range. Although a predetermined error range of the interval between peaks is ±150 msec., for example, this is not restrictive. FIG. 4 illustrates an example detected by the controller 10, in which, from time t₁ to time t₂, a variance in interval between peaks of each pulse wave is within ±150 msec four times in a row.

When the controller 10 determines that a stable pulse wave is detected continuously for a predetermined number of times after the pulse wave measurement processing is started (Yes in step S101 in FIG. 5), it starts acquisition of pulse wave (step S102). In other words, the controller 10 acquires a pulse wave used for measuring the blood component. The time at which the acquisition of pulse wave is started is time t₃, for example, in FIG. 4. The controller 10 may store the pulse wave acquired in the aforementioned manner in the memory 20. The biological information measurement apparatus 1 starts acquisition of pulse wave when it determines that a stable pulse wave is detected continuously for a predetermined number of times, which facilitates prevention of false detection.

After starting the pulse wave acquisition, the controller 10 terminates the pulse wave acquisition when termination condition of pulse wave acquisition is satisfied. The termination condition may be a case in which a predetermined time is passed after the pulse wave acquisition is started. The termination condition may be a case in which a pulse wave for a predetermined number of pulses is acquired. The termination condition is not limited thereto, and other conditions may be set appropriately. In the example illustrated in FIG. 4, the controller 10 terminates acquisition of pulse wave at time t₄ at which a predetermined time (e.g. 8 sec. or 15 sec.) has passed after time t₃. In this manner, the process illustrated in FIG. 5 ends.

When the controller 10 determines that a stable pulse wave is not detected. continuously for a predetermined number of times after the pulse wave measurement processing is started (No in step S101 in FIG. 5), it determines whether a predetermined time has passed or not after occurrence of the predetermined event for starting the pulse wave measurement processing (step S103).

When the controller 10 determines that the predetermined time (e.g. 30 sec.) has not passed after occurrence of the predetermined event for starting the pulse wave measurement processing (No in step S103), the process illustrated in FIG. 5 proceeds to step S101.

On the other hand, when the controller 10 cannot detect a stable pulse wave even if a predetermined time has passed after occurrence of the predetermined event for starting the pulse wave measurement processing (Yes in step S103), the measurement processing ends automatically (time-out) and the process illustrated in FIG. 5 ends.

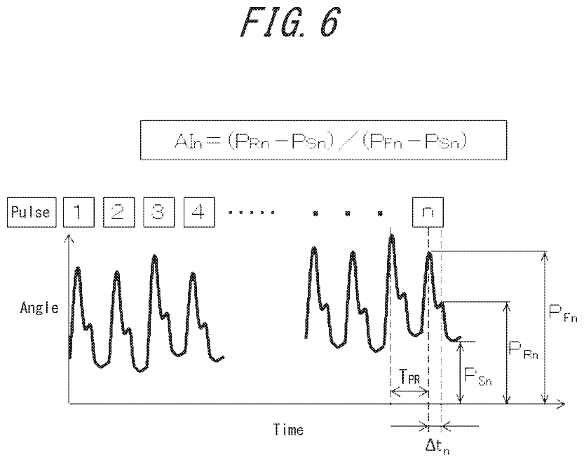

FIG. 6 is a diagram illustrating an example of a pulse wave acquired at the measured part (trunk) by using the biological information measurement apparatus 1. FIG. 6 illustrates an example in which the gyro sensor 12 is used as a detection means of pulsation. FIG. 6 illustrates the integral of the angular velocity acquired by an angular velocity sensor, which is the gyro sensor 12. In FIG. 6, the horizontal axis represents the time and the vertical axis represents the angle. Since the acquired pulse wave may include a noise caused by body motion of the subject, for example, a filter may be used to remove direct current (DC) component for compensation so as to extract pulsation components only.

The biological information measurement apparatus 1 calculates, from the acquired pulse wave, an index based on pulse wave, and measures the blood component by using the index based on pulse wave. A method of calculating an index based on pulse wave from an acquired pulse wave will be described with reference to FIG. 6. Pulse wave propagation is a phenomenon in which pulsation caused by blood pumped out of the heart is transmitted through the arterial walls or blood. The pulsation caused by the blood pumped out of the heart reaches, as a progressive wave, the ends of human limbs, and a part thereof is reflected from the blood vessel branches, blood vessel diameter changed portions, and the like, and is returned as a reflected wave. The index based on pulse wave includes, for example, a pulse wave velocity (PWV) of the progressive wave, a magnitude of reflected wave of pulse wave P_{R}, a difference in time Δt between the progressive wave and the reflected wave of the pulse wave, an augmentation index (AI) represented by a ratio of the progressive wave magnitude and the reflected wave magnitude of the pulse wave, and the like.

The pulse wave illustrated in FIG. 6 is a pulse of a user for n times (n is an integer of 1 or more). The pulse wave is a synthesized wave of a progressive wave caused by pumping blood out of the heart and a reflected wave generated from blood vessel branches or blood vessel diameter changed portions overlapping each other. In FIG. 6, P_{Fn} represents a peak magnitude of pulse wave by progressive wave with respect to each pulse, P_{Rn} represents a peak magnitude of pulse wave by reflected wave with respect to each pulse, and P_{Sn} is a minimum value of pulse wave with respect to each pulse. In FIG. 6, T_{PR} is an interval between peaks of pulse.

The index based on pulse wave includes those acquired by quantifying the information acquired from pulse wave. For example, PWV, which is one of indices based on pulse wave is calculated on the basis of a difference in propagation times between pulse waves measured at two measured parts such as an upper arm and an ankle, and a distance between the two parts. Specifically, PWV is calculated by acquiring pulse waves at two points of artery (e.g. upper arm and ankle) by synchronizing them, and dividing the difference in distance between two points (L) by the difference in time between pulse waves at two points (PTT). For example, as a magnitude of reflected wave P_{R}, which is one of indices based on pulse wave, a magnitude of peak of pulse wave by reflected wave P_{Rn} may be calculated, or P_{Rave}, which is an average of n times, may be calculated. For example, as a difference in time Δt between the progressive wave and the reflected wave of the pulse wave, which is one of indices based on pulse wave, a difference in time Δtₙ between predetermined pulses, or Δtₐᵥₑwhich is an averaged difference in time for n times may be calculated. For example, AI, which is one of indices based on pulse wave, is obtained by dividing the magnitude of reflected wave by the magnitude of progressive wave, and is represented by Aₙ=(P_{Rn}−P_{Sn})/(P_{Fn}−P_{Sn}). AIₙ represents AI with respect to each pulse. For example, AI may be an index based on pulse wave, which is obtained by measuring the pulse wave for a few seconds and calculating the average value of AIₙ (n is an integer of 1 to n) with respect to each pulse, AIₐᵥₑ.

The pulse wave propagation velocity PWV, the reflected wave magnitude PR, the difference in time Δt between the progressive wave and the reflected wave and AI vary depending on the stiffness of the blood vessel wall, and thus can be used for estimating the state of arteriosclerosis. For example, when the blood vessel walls are stiff, the pulse wave propagation velocity PWV is increased. For example, when the blood vessel walls are stiff, the reflected wave magnitude P_{R} is increased. For example, when the blood vessel walls are stiff, the difference in time Δt between the progressive wave and the reflected wave is decreased. For example, when the blood vessel walls are stiff, AI is increased. Furthermore, the biological information measurement apparatus 1 can estimate the state of arteriosclerosis and further can estimate the blood fluidity (viscosity) by using these indices based on pulse wave. In particular, the biological information measurement apparatus 1 can estimate the change in the blood fluidity on the basis of the change in the index based on pulse wave acquired from the same measured part of the same subject and acquired in a period of time (e.g. a few days) in which the state of arteriosclerosis remains almost the same. In this context, the blood fluidity indicates the flowability of blood. For example, when the blood fluidity is low, the pulse wave propagation velocity PWV is decreased. For example, when the blood fluidity is low, the magnitude of reflected wave P_{R} is decreased. For example, when the blood fluidity is low, the difference in time Δt between the progressive wave and the reflected wave is increased. For example, when the blood fluidity is low, AI is decreased.

In an embodiment, as an example of an index based on pulse wave, an example in which the biological information measurement apparatus 1 calculates the pulse wave propagation velocity PWV, the magnitude of the reflected wave P_{R}, the difference in time Δt between the progressive wave and the reflected wave, and AI is given. However, the index based on pulse wave is not limited thereto. For example, the biological information measurement apparatus 1 may use the posterior systolic blood pressure as an index based on pulse wave.

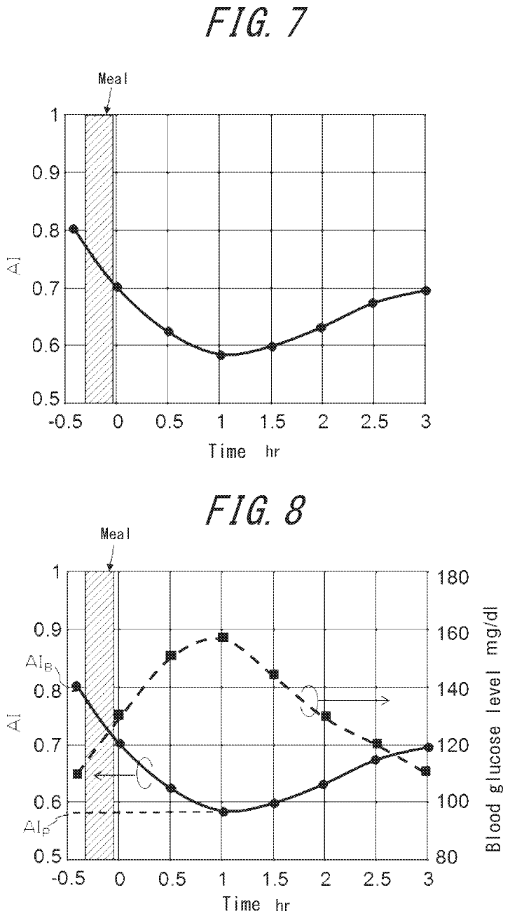

FIG. 7 is a diagram illustrating a change in the calculated AIs over time. In an embodiment, the pulse waves were acquired for about five seconds by using the biological information measurement apparatus 1 provided with an angular velocity sensor. The controller 10 calculated AIs with respect to each pulse from the acquired pulse waves, and then calculated the average AIₐᵥₑ. In an embodiment, the biological information measurement apparatus 1 acquired pulse waves at timings before and after the meal, and calculated the AI average value (hereinafter referred to as AI) as an example of an index based on acquired pulse wave. In FIG. 7, the horizontal axis represents an elapse of time defining the first measurement time after the meal as 0. The vertical axis in FIG. 7 represents AI calculated from the pulse wave acquired at that time.

The biological information measurement apparatus 1 acquired a pulse wave before the meal, immediately after the meal and every 30 minutes after the meal, and calculated a plurality of AIs on the basis of each pulse wave. The AI calculated from the pulse wave acquired before the meal was about 0.8. The AI immediately after the meal was smaller than that before the meal, and was the minimum extreme value in about one hour after the meal. Then the AI was gradually increased by the time the measurement was finished in three hours after the meal.

The biological information measurement apparatus 1 can estimate a change in blood fluidity from the calculated change in AI. For example, when the red blood cells, the white blood cells and the platelets in the blood agglutinate into clumps, or become more cohesive, the blood fluidity is lowered. For example, when the plasma water content in the blood is decreased, the blood fluidity is lowered. These changes in the blood fluidity depend on the health conditions of the subject such as, for example, glycolipid, heatstroke, dehydration, hypothermia and the like described later. The subject can notice a change of its own blood fluidity by using the biological information measurement apparatus 1 according to an embodiment before his/her health condition becomes severe. A decrease in the blood fluidity after the meal, a decrease in the blood fluidity to the lowest level in about one hour after the meal and a gradual increase in the blood fluidity thereafter can be estimated on the basis of the change in AI before and after the meal, illustrated in FIG. 7. The biological information measurement apparatus 1 may notify the state in which the blood fluidity is low and the state in which the blood fluidity is high. For example, the biological information measurement apparatus 1 may determine that the blood fluidity is low or high on the basis of the average value of AI of the subject's actual age. The biological information measurement apparatus 1 may determine that the blood fluidity is high if the calculated AI is larger than the average value or that the blood fluidity is low if the calculated AI is smaller than the average value. The biological information measurement apparatus 1 may determine that the blood fluidity is low or high on the basis of the AI before the meal. The biological information measurement apparatus 1 may estimate the degree of low blood fluidity on the basis of comparison between AI after the meal and AI before the meal, for example. The biological information measurement apparatus 1 may use the AI before the meal, that is, the fasting AI, as an index of the blood vessel age of the subject (stiffness of the blood vessel). An estimate error due to the blood vessel age (stiffness of the blood vessel) of the subject can be decreased if the biological information measurement apparatus 1 calculates an amount of change in the calculated AIs on the basis of the AI before the meal, that is, fasting AI, of the subject, for example. The biological information measurement apparatus 1 can estimate a change in the blood fluidity with higher accuracy.

FIG. 8 is a diagram illustrating the calculated AI and the measurement results of the blood glucose level. The pulse wave acquisition method and the AI calculation method are the same as the embodiment illustrated in FIG. 7. The vertical axis on the right side of FIG. 8 represents the blood glucose level and the vertical axis on the left side represents the calculated AI. The solid line in FIG. 8 represents the AI calculated from the acquired pulse wave and the dotted line represents the measured blood glucose level. The blood glucose level was measured immediately after the acquisition of pulse wave. The blood glucose level was measured by using a Medisafe FIT@ blood glucose meter from Terumo Corporation. The blood glucose level immediately after the meal was increased by about 20 mg/dl than that before the meal. The blood glucose level reached the maximum extreme value in one hour after the meal. Thereafter the blood glucose level was gradually decreased until the end of the measurement and returned to almost the same blood glucose level before the meal in about three hours after the meal.

As illustrated in FIG. 8, the blood glucose levels before and after the meal are negatively correlated to the AI calculated from the pulse wave. When the blood glucose level is increased, the glucose in the blood causes the white blood cells and the platelets in the blood to agglutinate into clumps or to become more cohesive, and consequently the blood fluidity may be lowered. When the blood fluidity is lowered, the pulse wave propagation velocity PWV may be decreased. When the pulse wave propagation velocity PWV is decreased, the difference in time Δt between the progressive wave and the reflected wave may be increased. When the difference in time Δt between the progressive wave and the reflected wave is increased, the magnitude of the reflected wave P_{R}, may become smaller than that of the progressive wave P_{F}. If the magnitude of the reflected wave P_{R} is smaller than that of the progressive wave P_{F}, AI may be decreased. The AI within a few hours after the meal (in an embodiment, within three hours) is correlated to the blood glucose level, and thus a change in the blood glucose level of the subject can be estimated from the change in AI. When the blood glucose level of the subject is measured and the correlation with AI is acquired in advance, the biological information measurement apparatus 1 can estimate the blood glucose level of the subject from the calculated AI.

On the basis of the time at which AIₚ, the minimum extreme value of AI detected first after the meal, occurs, the biological information measurement apparatus 1 can estimate the glucose metabolism status of the subject. The biological information measurement apparatus 1 estimates the blood glucose level, for example, as the glucose metabolism status. As an example of estimating the glucose metabolism status, when AIp, which is the minimum extreme value of AI detected first after the meal, is detected after the elapse of a predetermined time or more (e.g. an hour and a half after the meal), for example, the biological information measurement apparatus 1 can estimate that the subject has impaired glucose metabolism (diabetic patient).

On the basis of the difference between AI_{B}, which is AI before the meal, and AIp, which is the minimum extreme value of AI detected first after the meal, (AI_{B}−AIp), the biological information measurement apparatus 1 can estimate the glucose metabolism status of the subject. As an example of estimating the glucose metabolism status, for example, when (AI_{B}−AIp) is a predetermined value or more (e.g. 0.5 or more), the subject is assumed to be an impaired glucose metabolism (postprandial hyperglycemia).

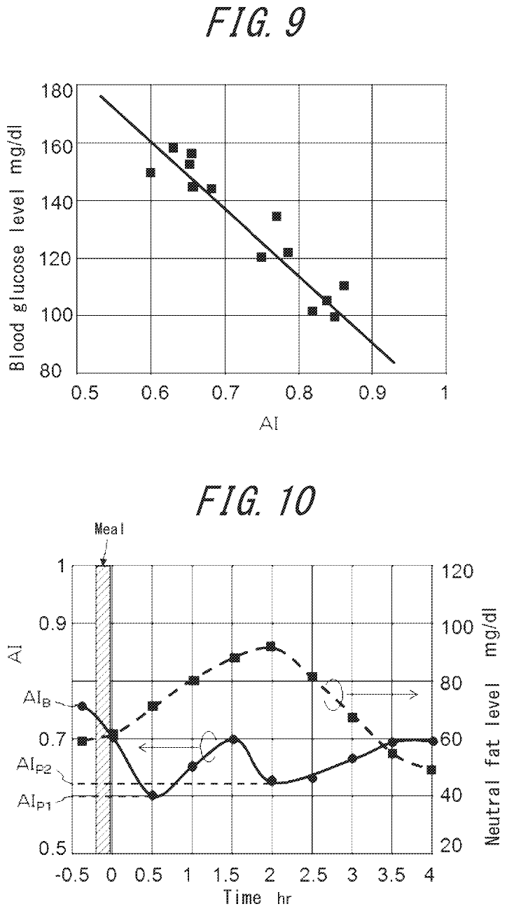

FIG. 9 is a diagram illustrating a relationship between the calculated AI and. the blood glucose level. The calculated AI and the blood glucose level were acquired within one hour after the meal, which is a period in which the blood glucose level changes a lot. The data of FIG. 9 includes those acquired from the same subject after some different meals. As illustrated in FIG. 9, the calculated AI and the blood glucose level are negatively correlated to each other. The correlation coefficient between the calculated AI and the blood glucose level was 0.9 or more. For example, if the correlation between the calculated AI and the blood glucose level as illustrated in FIG. 9 is acquired with respect to each subject in advance, the biological information measurement apparatus 1 can also estimate the blood glucose level of the subject from the calculated AI.

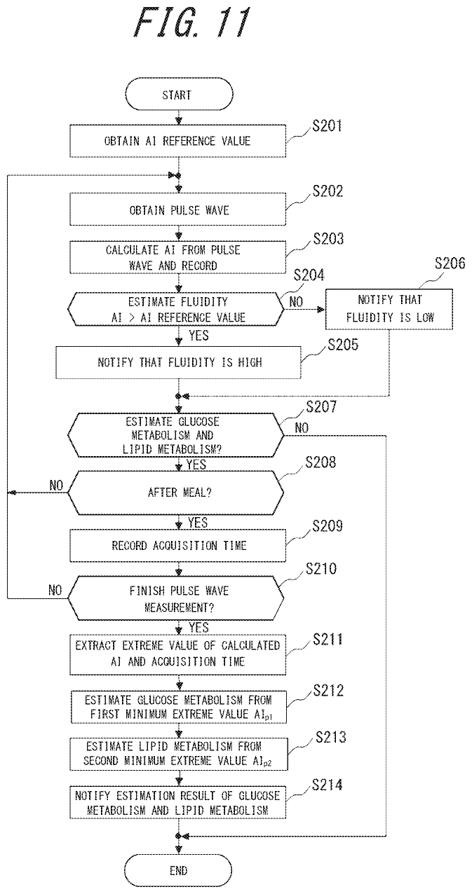

FIG. 10 is a diagram illustrating the calculated AI and the measurement result of neutral fat level. The pulse wave acquisition method and the AI calculation method are the same as those illustrated in the embodiment in FIG. 7. In FIG. 10, the vertical axis on the right side represents the neutral fat level in the blood, and the vertical axis on the left side represents AI. In FIG. 10, the solid line represents the AI calculated from the acquired pulse wave and the dotted line represents the measured neutral fat level. The neutral fat level was measured immediately after acquisition of the pulse wave. The neutral fat level was measured by using a lipid measurement apparatus, “POCKET LIPID,” from Techno Medica Co., Ltd. Compared with the neutral fat level before the meal, the maximum extreme value of the neutral fat level after the meal was increased by about 30 mg/dl. The neutral fat level reached the maximum extreme value in about two hours after the meal. Thereafter the neutral fat level was gradually decreased until the end of the measurement, and returned to almost the same neutral fat level before the meal in about three and a half hours after the meal.

On the other hand, as to the minimum extreme value of the calculated AI, the first minimum extreme value AI_{P1} was detected in 30 minutes after the meal, and the second minimum extreme value AI_{P2} was detected in two hours after the meal. The first minimum extreme value AI_{P1} detected in 30 minutes after the meal may be influenced by the aforementioned blood glucose level after the meal. The second minimum extreme value AI_{P2} was detected in about two hours after the meal and the maximum extreme value of the neutral fat was detected in about two hours after the meal. Thus these values occurred almost at the same time. From mentioned above, it is estimated that the second minimum extreme value AI_{P2} detected in a predetermined time or later after the meal is influenced by the neutral fat, As with the blood glucose level, it was found that the neutral fat level before and after the meal was negatively correlated to the AI calculated from the pulse wave. In particular, AI_{P2}, which is the minimum extreme value of AI detected in a predetermined time or later after the meal (in an embodiment, about one and a half hours or later) is correlated to the neutral fat level, thus a change in the neutral fat level of the subject can be estimated on the basis of the change in AI. Further, if the neutral fat level is measured and correlation with AI is acquired in advance, the biological information measurement apparatus 1 can estimate the neutral fat level of the subject from the calculated AI.

On the basis of the time at which the second minimum extreme value AI_{P2}, which is detected in a predetermined time or later after the meal, occurs, the biological information measurement apparatus 1 can estimate the lipid metabolism status of the subject. The biological information measurement apparatus 1 estimates the lipid level, for example, as the lipid metabolism status. As an example of estimating the lipid metabolism status, when the second minimum extreme value AI_{P2} is detected in a predetermined time or later (e.g. four hours or later) after the meal, the biological information measurement apparatus 1 can estimate that the subject is impaired lipid metabolism (hyperlipidemia).

On the basis of the difference between AI_{B}, which is the AI before the meal, and AIp₂, which is the second minimum extreme value detected in a predetermined time or later after the meal, (AI_{B}−AIp₂), the biological information measurement apparatus 1 can estimate the lipid metabolism status of the subject. As an example of estimating the impaired lipid metabolism status, for example, when (AI_{B}−AIp₂) is 0.5 or more, the biological information measurement apparatus 1 can estimate that the subject is an impaired lipid metabolism (postprandial hyperlipidemia).

Furthermore, on the basis of the measurement results illustrated in FIGS. 8 to 10, the biological information measurement apparatus 1 according to an embodiment can estimate the glucose metabolism status of the subject on the basis of the first minimum extreme value AIp₁ detected first after the meal and its occurrence time. Moreover, the biological information measurement apparatus 1 according to an embodiment can estimate the lipid metabolism status of the subject on the basis of the second minimum extreme value AIp₂ detected in a predetermined time or later after the first minimum extreme value AIp₁ and its occurrence time.

In an embodiment, although the neutral fat was taken as an example of estimating the lipid metabolism, it is not restrictive. The lipid level estimated by the biological information measurement apparatus 1 includes, for example, total cholesterol, HDL cholesterol, LDL cholesterol and the like. These lipid levels exhibit the tendency similar to that of the aforementioned neutral fat.

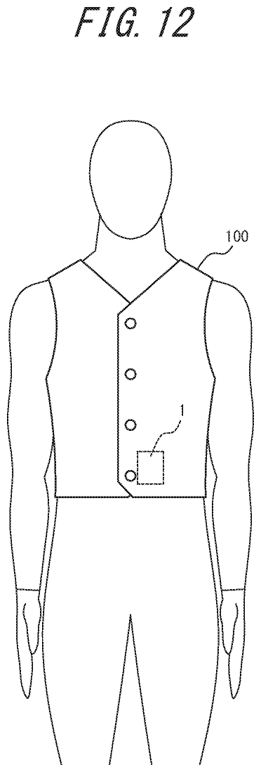

FIG. 11 is a flowchart illustrating a procedure of estimating the blood fluidity and the status of glucose metabolism and lipid metabolism on the basis of AI. A process flow of estimating the blood fluidity and the status of glucose metabolism and lipid metabolism by the biological information measurement apparatus 1 according to an embodiment will be explained with reference to FIG. 11.

As illustrated in FIG. 11, the biological information measurement apparatus 1 acquires an AI reference value of the subject as a default (step S201). As the AI reference value, an average AI estimated from the age of the subject or the fasting AI of the subject acquired in advance may be used. Further, the biological information measurement apparatus 1 may use the AI determined as measured before the meal in steps S202 to S208 or the AI calculated immediately before the measurement of pulse wave as the AI reference value. In this case, the biological information measurement apparatus 1 executes step S201 after steps S202 to S208.

Subsequently, the biological information measurement apparatus 1 acquires a pulse wave (step S202). For example, the biological information measurement apparatus 1 determines whether or not the pulse wave acquired during a predetermined measurement time period (e.g. for five seconds) is a predetermined. amplitude or more. If the acquired pulse wave is the predetermined amplitude or more, the process proceeds to step S203. If the acquired pulse wave is not the predetermined amplitude or more, the process repeats step S202 (these steps are not illustrated). In step S202, for example, when the biological information measurement apparatus 1 detects a pulse wave of the predetermined amplitude or more, it automatically acquires a pulse wave.

The biological information measurement apparatus 1 calculates, from the pulse wave acquired in step S202, the AI as an index based on pulse wave, and stores the AI in the memory 20 (step S203). As the AI, the biological information measurement apparatus 1 may calculate the average AIₐᵥₑ from AIₙ (n is an integer of 1 to n) with respect to each predetermined number of pulses (e.g. three pulses). Alternatively, the biological information measurement apparatus 1 may calculate the AI with respect to a specific pulse.

AI may be compensated by the number of pulses P_{R}, the pulse pressure (P_{F}−P_{S}), the body temperature, the temperature of the measured part and the like. It is known that the pulse and the AI and the pulse pressure and the AI are negatively correlated to each other, respectively. It is also known that the temperature and the AI are positively correlated to each other. When compensation is performed, the biological information measurement apparatus 1 calculates, in addition to AI, the pulse and the pulse pressure in step S203, for example. The biological information measurement apparatus 1 may include a temperature sensor along with the gyro sensor 12 to acquire the temperature of the measured part during pulse wave acquisition in step S202. Through substitution of the acquired pulse, the pulse pressure, the temperature, and the like in a compensation formula prepared in advance, the biological information measurement apparatus 1 compensates the AI.

Subsequently the biological information measurement apparatus 1 compares the AI reference value acquired in step S201 with the AI calculated in S203 to estimate the blood fluidity of the subject (step S204). When the calculated AI is larger than the AI reference value (YES), it is estimated that the blood fluidity is high. In this case, the biological information measurement apparatus 1 notifies that the blood fluidity is high, for example (step S205). When the calculated AI is not larger than the AI reference value (NO), it is estimated that the blood fluidity is low. In this case, the biological information measurement apparatus 1 notifies that the blood fluidity is low, for example (step S206).

Subsequently, the biological information measurement apparatus 1 confirms with the subject whether or not to estimate the status of glucose metabolism and lipid metabolism (step S207). If the glucose metabolism and the lipid metabolism are not estimated in step S207 (NO), the process ends. If the glucose metabolism and the lipid metabolism are estimated in step S207 (YES), the biological information measurement apparatus 1 confirms whether the calculated AI was acquired before or after the meal (step S208). If the AI is acquired not after the meal (acquired before the meal) (NO), the process returns to step S202 to acquire the next pulse wave. If the AI is acquired after the meal (YES), the biological information measurement apparatus 1 stores the acquisition time of the pulse wave corresponding to the calculated AI (step S209). When the pulse wave is acquired subsequently (NO in step S210), the process returns to step S202 and the biological information measurement apparatus 1 acquires the next pulse wave. When the measurement of pulse wave is finished (YES in step S210), the process proceeds to and after step S211, and the biological information measurement apparatus 1 estimates the status of glucose metabolism and lipid metabolism of the subject.

Subsequently, the biological information measurement apparatus 1 extracts the minimum extreme value and the time thereof from a plurality of AIs calculated in step S204 (step S211). For example, when the AI as represented by the solid line in FIG. 10 is calculated, the biological information measurement apparatus 1 extracts the first minimum extreme value AIp₁ measured in 30 minutes after the meal and the second minimum extreme value AIp₂ measured in two hours after the meal.

Subsequently, the biological information measurement apparatus 1 estimates the glucose metabolism status of the subject from the first minimum extreme value AIp₁ and the time thereof (step S212). The biological information measurement apparatus 1 further estimates the lipid metabolism status of the subject from the second minimum extreme value AIp₂ and the time thereof (step S213). An example of estimating the status of glucose metabolism and lipid metabolism of the subject is the same as the aforementioned example illustrated in FIG. 10, and thus is omitted.

Subsequently, the biological information measurement apparatus 1 notifies the estimation results of steps S212 and S213 (step S214), and the process illustrated in FIG. 11 ends.

The notification is given by the audio output interface 16. The audio output interface 16 gives notification through voice message such as, for example, “glucose metabolism is normal,” “glucose metabolism disorder is suspected,” “lipid metabolism is normal,” “lipid metabolism disorder is suspected” and the like. The audio output interface 16 may also give an advice such as “go to see a doctor,” “dietary review needed” and the like. Then, the process illustrated in FIG. 11 ends. As an audio output interface that gives notification through voice message, an audio system may be used. The audio system is installed in advance in an external apparatus that communicates wired or wirelessly with the biological information measurement apparatus 1 provided to the garment according to the present embodiment. The audio signal from the biological information measurement apparatus 1 may be input to AUX terminal of the audio system through an AUX cable in wired communication. The audio signal may also be sent from the biological information measurement apparatus 1 to the audio system through any wireless communication such as FM transmitter, Bluetooth® and the like. Moreover, a dedicated audio output interface that outputs audio from the biological information measurement apparatus 1 may be provided outside.

Instead of or along with the aforementioned notification through voice message, a notification may be given through display on the display 14. It is to be noted that, as a display that gives notification through display, a display provided to an external apparatus that communicates wired or wirelessly with the biological information measurement apparatus 1 provided to the garment according to the present embodiment may be used. Furthermore, a dedicated display that displays a notification from the biological information measurement apparatus 1 may be provided outside the apparatus.

The biological information measurement apparatus 1 may also allow the audio output interface 16 to output a sound indicating that the gyro sensor 12 is detecting a motion factor. This enables the subject to know easily and reliably that the gyro sensor 12 detects a motion factor correctly in the biological information measurement apparatus 1.

As described above, the biological information measured by the biological information measurement apparatus 1 may include the information relating to at least one of the pulse wave, the pulse, the breathing, the beating, the pulse wave propagation velocity and the blood flow rate of the wearer, which is a subject.

Further, on the basis of the biological information measured by the biological information measurement apparatus 1, the controller 10 may estimate at least one of the physical condition, the drowsiness, the sleeping, the wakefulness, the psychological state, the physical state, the feeling, the mind and body condition, the mental condition, the autonomic nerve, the state of stress, the consciousness state, the blood component, the asleep condition, the breathing condition and the blood pressure of the wearer, which is a subject. In this context, the “physical state” of the subject may include, for example, presence or absence of symptoms of heatstroke, fatigue, attitude sickness, diabetes, metabolic syndrome, and the like, degree of these symptoms and presence of absence of a sign of these symptoms. Further, the blood component may be neutral fat, blood glucose level and the like,

Next, an example of configuration of the garment provided with the biological information measurement apparatus 1 according to the present embodiment will be described. It is to be noted that, when the biological information of the wearer of the garment, which is a subject, is measured, arrangement of the gyro sensor 12 is important, and the gyro sensor 12 may be arranged in any positions of the biological information measurement apparatus 1 only if it can operate in accordance with the control of the controller 10. Thus, arrangement of the gyro sensor 12 will be described in detail below, and description of the other configuration of the biological information measurement apparatus 1 will be omitted. Hereinafter an illustration is given assuming that the biological information measurement apparatus 1 includes at least the gyro sensor 12.

FIG. 12 is an example of configuration of the garment provided with the biological information measurement apparatus 1. In FIG. 12, the garment 100 is a top (a jacket or an upper wear). In FIG. 12, the garment 100 is a vest (waistcoat). The wearer wears it by slipping his/her arms into sleeves.

As illustrated in FIG. 12, the garment 100, as a top, includes the biological information measurement apparatus 1 provided with the gyro sensor 12 on the back side thereof opposing the abdomen of the wearer. In FIG. 12, the biological information measurement apparatus 1 is illustrated by the dotted line, which indicates that the biological information measurement apparatus 1 is arranged on the back side of the garment 100. In the state illustrated in FIG. 12, the biological information measurement apparatus 1 is located between the measured part on the abdomen of the wearer, which is a subject, and the garment 100. In this manner, in a state in which the wearer, which is a subject, wears the garment 100, at least a part of the biological information measurement apparatus 1 abuts near the abdomen of the wearer. In this context, as illustrated in FIGS. 3A and 3B, the biological information measurement apparatus 1 is configured such that the abutment 40 thereof abuts the abdomen, which is a measured part, of the wearer. In this manner, the gyro sensor 12 provided to the biological information measurement apparatus 1 can detect a change in the abdomen of the wearer of the garment 100.

FIG. 12 illustrates an example in which the garment 100, which is a vest (waistcoat), is an open-front type, with buttons on the front side of the wearer. However, the garment 100 according to the present embodiment is not limited to the open-front type garment, and may be an open-back or an open-side type garment.

The garment 100 according to the present embodiment is not limited to the button-down type as illustrated in FIG. 12, and may be a jip up type provided with a fastener, for example. Furthermore, the garment 100 according to the present embodiment may be neither the button-down type nor the jip up type, and may be a pullover type made of a stretch material.

Further, in FIG. 12, the garment 100 is a vest (waistcoat). However, the garment 100 according to the present embodiment is not limited to the garment such as a vest (waistcoat), and may be a variety of types of outerwear, For example, the garment 100 according to the present embodiment may be a garment such as a shirt including a T-shirt, a dress shirt and a polo shirt, a blouse or cut and sewn. The garment 100 according to the present embodiment may also be underwear such as a tank top or a camisole. Furthermore, the garment 100 according to the present embodiment may be a jacket, a jumper, a sweater, a cardigan or a sweatshirt. Moreover, the garment 100 according to the present embodiment may be a wetsuit closely contact the body of the wearer. The garment 100 according to the present embodiment may also be a life jacket or a life vest.

The garment 100 according to the present embodiment may be a variety of types of tops described above. In light of allowing the gyro sensor 12 of the biological information measurement apparatus 1 to abut the measured part of the wearer, which is a subject, the garment 100 according to the present embodiment may preferably be a garment configured to keep a state in which the garment abuts the measured part of the wearer at least in a closely contacting manner when being worn. However, in the garment 100 according to the present embodiment, it is not necessarily mean that the gyro sensor 12 should always abut the measured part of the wearer when being worn. In this case, when the biological information is measured by the garment 100, the wearer pushes the biological information measurement apparatus 1 against his/her body from outside of the garment 100 so that the gyro sensor 12 abuts the measured part.

In this manner, the garment 100 according to the present embodiment includes at least the gyro sensor 12 and preferably the controller 10. The gyro sensor 12 detects a change in the measured part (e.g. abdomen) of the wearer of the garment 100. The controller 10 then performs the measurement processing of the biological information of the wearer, which is a subject, on the basis of the detected change. Specifically, the gyro sensor 12 may detect a motion factor caused by the change in the abdomen of the wearer. The controller 10 may also perform the measurement processing of the biological information of the wearer, which is a subject, on the basis of the detected motion factor. According to the garment 100 of the present embodiment, the biological information of the wearer of the garment 100 can be easily measured.

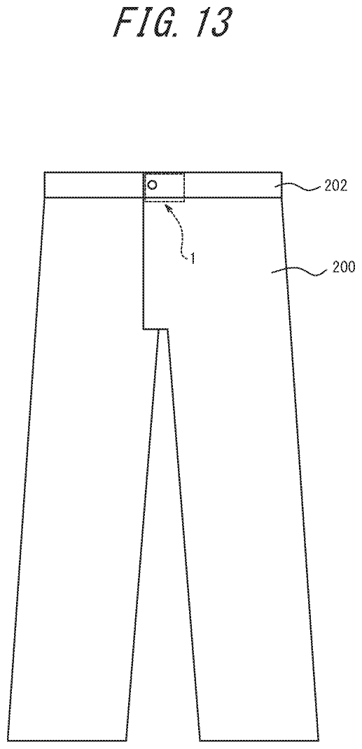

FIG. 13 is another configuration example of the garment provided with the biological information measurement apparatus 1. In FIG. 13, the garment 200 is a bottom (a lower wear). In FIG. 13, the garment 200 is long trousers (pants). The wearer wears it by slipping his/her legs into them.

As illustrated in FIG. 13, the garment 200 as a bottom includes the biological information measurement apparatus 1 provided with the gyro sensor 12 on the back side of the waist-band 202 surrounding the abdomen of the wearer, In FIG. 13, the biological information measurement apparatus 1 is illustrated by the dotted line, which indicates that the biological information measurement apparatus 1 is arranged on the back side of the garment 200. In the state illustrated in FIG. 13, the biological information measurement apparatus 1 is located between the measured part around the abdomen of the wearer, which is a subject, and the garment 200. In this manner, in a state in which the wearer, which is a subject, wears the garment 200, at least a part of the biological information measurement apparatus 1 abuts near the abdomen of the wearer. In this context, as illustrated in FIG. 3A or FIG. 3B, the biological information measurement apparatus 1 is configured such that the abutment 40 abuts the abdomen, which is a measured part, of the wearer. In this manner, the gyro sensor 12 provided to the biological information measurement apparatus 1 can detect a change in the abdomen of the wearer of the garment 200.

In FIG. 13, the garment 200, which is long trousers (pants), has buttons and a fastener on the front side of the wearer and is an open-front type. However, in the garment 200 according to the present embodiment, the waist-band 202 may be made of a stretch material such as rubbers, for example, and may have no buttons or fasteners.

In FIG. 13, although the garment 200 is long trousers (pants), the garment 200 according to the present embodiment is not limited to the garment such as long trousers (pants), and it may be a variety of types of bottoms. For example, the garment 200 according to the present embodiment may be bottoms such as slacks, jeans, riding breeches, training pants, sweat pants, half pants or short pants. The garment 200 according to the present embodiment is not limited to bottoms such as pants, and may be bottoms such as skirt, spat, stirrup legging or tights. Compared with the garment 100 like tops, the garment 200 of bottoms includes the waist-band 202. The waist-band 202 is usually brought tight contact with the wearer, which is a subject. In this manner, the garment 200 according to the present embodiment can measure the biological information of the wearer, which is a subject, in a relatively accurate manner in a relatively large number of occasions.

In this manner, the garment 200 according to the present embodiment includes at least the gyro sensor 12 and preferably the controller 10. The gyro sensor 12 detects a change in the measured part (e.g. abdomen) of the wearer of the garment 200, and the controller 10 then performs the measurement processing of the biological information of the wearer, which is a subject, on the basis of the detected change. In this context, when the garment 200 according to the present embodiment is a bottom, it may be supported at a waist of the wearer, which is a subject. When the garment 200 according to the present embodiment is a bottom, the gyro sensor 12 may be provided to the waist-band 202.

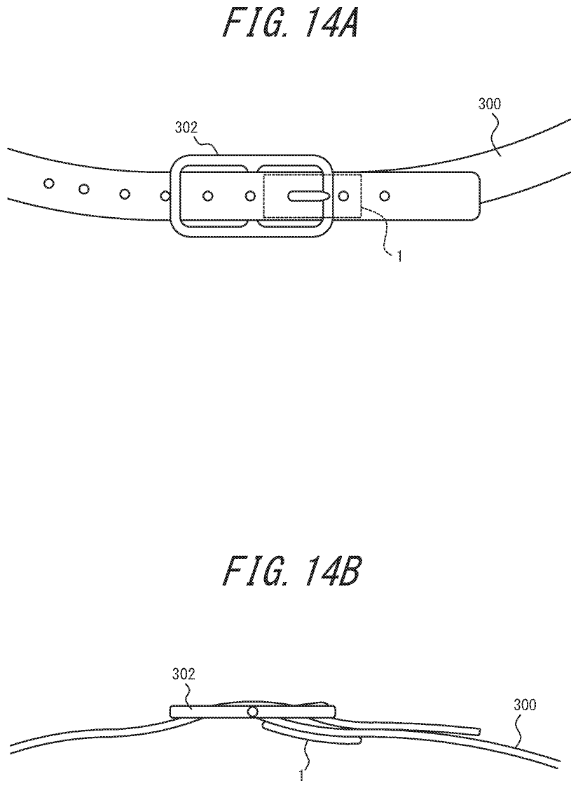

FIG. 14 is a diagram illustrating a still another example of the garment provided with the biological information measurement apparatus 1. In FIG. 14, the garment 300 is a belt worn by the wearer. FIG. 14 illustrates an example in which the garment 300 is a belt that is passed through the belt loops for pants, for example.

As illustrated in FIG. 14A, the garment 300 as a belt includes a buckle 302 at a waist band portion surrounding the abdomen of the wearer. Further, as illustrated in FIG. 14B, the garment 300 includes the biological information measurement apparatus 1 provided with the gyro sensor 12 on the back side of at least a part of at least one of the waist band and the buckle 302. In FIG. 14A, the biological information measurement apparatus 1 is illustrated by a dotted line, which indicates that the biological information measurement apparatus 1 is arranged on the back side of the garment 300. In a state illustrated in FIG. 14, the biological information measurement apparatus 1 is located between the measured part around the abdomen of the wearer, which is a subject, and the garment 300. Therefore, in a state in which the wearer, which is a subject, wears the garment 300, at least a part of the biological information measurement apparatus 1 abuts near the abdomen of the wearer. In this context, the biological information measurement apparatus 1 is configured such that the abutment 40 abuts the abdomen, which is a measured part of the wearer, as illustrated in FIG. 3A or FIG. 3B. With this configuration, the gyro sensor 12 provided to the biological information measurement apparatus 1 can detect a change in the abdomen of the wearer of the garment 300.

The garment 300, which is a belt, is usually brought tight contact with the wearer, which is a subject. In this manner, the garment 300 according to the present embodiment can also measure the biological information of the wearer, which is a subject, in a relatively accurate manner in a relatively large number of occasions. As another example, the garment 300 may also be suspenders, for example.

In this manner, the garment 300 according to the present embodiment includes at least the gyro sensor 12 and preferably the controller 10. The gyro sensor 12 detects a change in the measured part (e.g. abdomen) of the wearer of the garment 300, and the controller 10 then performs the measurement processing of the biological information of the wearer, which is a subject, on the basis of the detected change. In this context, the garment 300 according to the present embodiment may be a belt, for example, by which the wearer, which is a subject, supports his/her outfit. When the garment 300 according to the present embodiment is a belt, for example, the outfit of the wearer may be supported at the waist of the wearer. In this context, in the garment 300 according to the present embodiment, the gyro sensor 12 may be provided to the buckle 302 of the belt.

As described above, in the garment 100, 200 or 300 according to the present embodiment, the gyro sensor 12 may be provided to a position that comes in contact with the wearer of the garment 100, 200 or 300. For example, when the gyro sensor 12 is located at a position which comes in contact with the measured part of the wearer, which is a subject, the biological information measurement apparatus 1 can accurately measure the biological information of the subject.

Further, in the garment 100, 200 or 300 according to the present embodiment, the gyro sensor 12 may be configured to be detachable. According to the above described configuration, for example, the gyro sensor 12 may be detached from the garment 100, 200 or 300 and cleaning of the garment 100, 200 or 300 is facilitated. Even if the garment 100, 200 or 300 or the gyro sensor 12 is lost or breaks down, with the above described configuration, it is not necessary to purchase both of them together.

In the garment 100, 200 and 300 according to the present embodiment, the position of the gyro sensor 12 may be changed. With this configuration, even if the gyro sensor 12 is not brought to abut the measured part in an appropriate manner, for example, the positional relationship between them can be easily adjusted. When the positional relationship between the gyro sensor 12 and the measured part is appropriately adjusted, the biological information measurement apparatus 1 can accurately measure the biological information of the wearer, which is a subject.

Further, in the garment 100, 200 or 300 according to the present embodiment, the gyro sensor 12 may be provided to inside or outside of the garment 100, 200 or 300. In the garment 100, 200 or 300 according to the present embodiment, an extremely variety of configurations is expected, and a variety of required measuring accuracies of the biological information is expected. Therefore, in this embodiment, the gyro sensor 12 may be provided to a variety of positions such as inside or outside of these garments, depending on the configuration of the garment and/or the required measuring accuracy of the biological information.

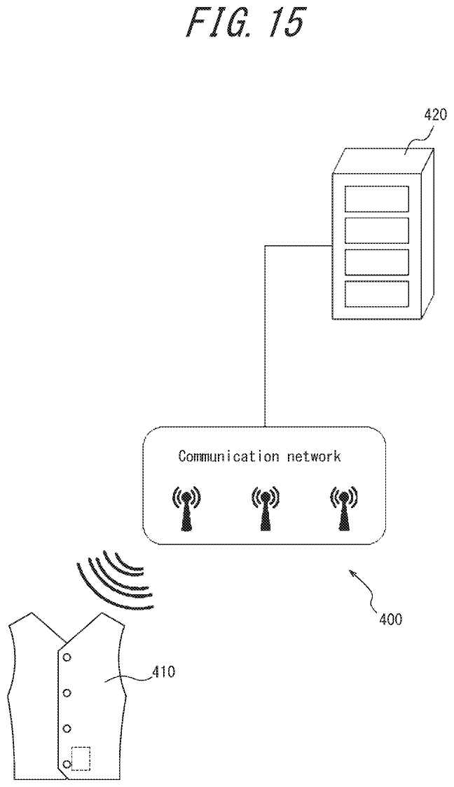

FIG. 15 is a diagram illustrating a schematic configuration of the biological information measurement system according to an embodiment. A biological information measurement system 400 according to an embodiment illustrated in FIG. 15 includes a garment 410, an external apparatus 420 and a communication network.

In the example of the biological information measurement system 400, the garment 410 is the top 100 as described above. However, the garment 410 may be a variety of types of garment, such as the above described bottom 200 or belt 300, worn by the wearer. The garment 410 detects a change in a predetermined part of the wearer of the garment 410. Thus the garment 410 is provided with the gyro sensor 12. The garment 410 also includes a communication interface (that can be connected wired or wireless), and sends a detected change in the abdomen of the wearer to the external apparatus 420. In the biological information measurement system 400, the external apparatus 420 performs a variety of kinds of operations relating to measurement of the biological information on the basis of the change in the abdomen of the wearer received. Thus the external apparatus 420 includes a variety of necessary function parts including a controller (e.g. a processor such as a CPU). Specifically, for example, the gyro sensor 12 of the garment 410 may detect a motion factor caused by a change in a predetermined part (e.g. abdomen) of the wearer of the garment 410. The external apparatus 420 may perform the measurement processing of the biological information on the basis of the motion factor received.

In FIG. 15, the garment 410 and the external apparatus 420 are assumed to be connected wirelessly. However, the biological information measurement system 400 is not limited to such connection. For example, the garment 410 and the external apparatus 420 may be connected by a predetermined cable, for example.

In this manner, the biological information measurement system 400 includes the garment 410 and the external apparatus 420. The garment 410 is provided with the gyro sensor 12. In this context, the gyro sensor 12 detects a motion factor caused by a change in a predetermined part (e.g. abdomen) of the wearer with the biological information measurement apparatus 1 abutted a predetermined part of the wearer. The external apparatus 420 also includes the above described controller. The external apparatus 420 may include an artificial intelligence function, a machine learning function, a deep learning function and the like, and may perform a variety of operations relating to the measurement of the biological information by using an algorithm acquired statistically on the basis of the motion factor received from the garment 410.

In order to disclose the embodiment of the present disclosure completely and clearly, some examples have been described. However, the appended claims are not limited to the above described embodiments, and are to be composed such that all modifications and alternative constructions that can be created, within the range of basic matters described in this specification, by those skilled in the art of this technical field. Moreover, each requirement indicated in some embodiments may be combined in any manner.

For example, in the embodiment of the present disclosure, the garment provided with the biological information measurement apparatus 1 ((the controller 10 and) the gyro sensor 12) and the biological information measurement system 400 have been described. However, the disclosed embodiment may be implemented as a biological information measurement method using the biological information measurement apparatus 1 provided with the gyro sensor 12. In this method, a change in a predetermined part of the wearer of the garment 100 is detected by the gyro sensor 12 provided to the garment 100. Further, in this method, the measurement processing of the biological information of the subject is performed on the basis of the change detected in the above described state. Specifically, for example, in the above described method, a motion factor caused by a change in a predetermined part (e.g. abdomen) of the wearer of the garment 100 may be detected by the gyro sensor 12 provided to the garment 100. Further, in the method, the measurement processing of the biological information of the wearer, which is a subject, may be performed on the basis of the motion factor detected.

Moreover, in the above described embodiment, although the biological information measurement apparatus 1 is provided with the abutment 40 and the support 50, for example, the biological information measurement apparatus 1 may not include the support 50. In this context, a part of an abutting surface of the biological information measurement apparatus 1 abuts the subject at a position that is different from a position of the measured part, and as a result the abutment 40 is kept abutting the measured part.

In the above embodiment, the abutment 40 is fixed to the biological information measurement apparatus 1. However, the abutment 40 is not necessarily fixed directly to the biological information measurement apparatus 1. The abutment 40 may be fixed to a holding tool that is used by being fixed to the biological information measurement apparatus 1.

### REFERENCE SIGNS LIST

1 Biological information measurement apparatus

10 Controller

11 Power source

12 Gyro sensor

14 Display

16 Audio output interface

17 Communication interface

18 Vibrator

19 Elastic member

20 Memory

40 Abutment

50 Support

100 Top (Upper wear)

200 Bottom (Lower wear)

202 Waist band

300 Belt

302 Buckle

400 Biological information measurement system

410 Garment

420 External apparatus

1. A garment, comprising:
a gyro sensor configured to detect a change in a wearer's abdomen; and
a controller configured to measure biological information of the wearer based on the change detected.

2. The garment according to claim 1, wherein the change includes at least one of a change caused by movement of a blood vessel of the wearer, a change caused by breathing of the wearer, and a change caused by body motion of the wearer.

3. The garment according to claim 2, wherein the blood vessel includes an aorta of the wearer.

4. The garment according to claim 3, wherein the aorta includes at least one of an abdomen aorta and a thoracic aorta of the wearer.

5. The garment according to claim 1, wherein the biological information includes information relating to at least one of pulse wave, pulse, breathing, beating, pulse wave propagation velocity and blood flow rate of the wearer

6. The garment according to claim 1, wherein the controller estimates, based on the biological information, information relating to at least one of physical condition, drowsiness, sleeping, wakefulness, psychological state, physical state, feeling, mind and body condition, mental condition, autonomic nerve, state of stress, consciousness state, blood component, sleeping condition, breathing condition, and blood pressure of the wearer.

7. The garment according to claim 1, wherein the gyro sensor is at a position that comes in contact with the wearer of the garment.

8. The garment according to claim 1, wherein the gyro sensor is detachable.

9. The garment according to claim 1, wherein a position of the gyro sensor can be changed.

10. The garment according to claim 1, wherein the gyro sensor is provided inside or outside of the garment.

11. The garment according to claim 1, wherein the garment is supported at a waist of the wearer.

12. The garment according to claim 1, wherein the gyro sensor is provided to a waist band.

13. The garment according to claim 1, wherein the garment is a belt by which the wearer supports the wearer's outfit.

14. The garment according to claim 13, wherein the outfit is supported at a waist of the wearer.

15. The garment according to claim 13, wherein the gyro sensor is provided to a buckle of the belt.

16. A biological information measurement method, the method comprising:
detecting a change in abdomen of a wearer of a garment by a gyro sensor provided to the garment; and
performing, based on the change detected, measurement processing of biological information of the wearer.

17. A biological information measurement system, comprising:
a garment including a gyro sensor configured to detect a change in a wearer's abdomen; and
an external apparatus including a controller configured to measure biological information of the wearer based on the change detected.
